(19) United States
(12) Patent Application Publication
(10) Pub. No.: US 2016/0272930 A1
(43) Pub. Date: Sep. 22, 2016

(54) ALGAE GROWTH SYSTEM AND METHOD

(71) Applicant: ALGALO INDUSTRIES LTD., Kibbutz Ein Hamifratz (IL)

(72) Inventor: Doron GAT, Kibbutz Ein HaChoresh (IL)

(73) Assignee: Algalo Industries Ltd., Kibbutz Ein HaMifratz (IL)

(21) Appl. No.: 15/028,943

(22) PCT Filed: Oct. 14, 2014
(86) PCT No.: PCT/IL2014/050900

§ 371 Date: Apr. 13, 2016

Related U.S. Application Data
(60) Provisional application No. 61/890,331, filed on Oct. 14, 2013.

(51) Int. Cl. C12M 1/00; C12M 1/02

(52) U.S. Cl. CPC C12M 29/06; C12M 21/02; C12M 41/08; C12M 41/24; C12M 23/22

(57) ABSTRACT

There is provided an Algae growth system which combines the advantages of closed systems with the simplicity and low costs of open systems, the system having a transparent photo-bioreactor device which has a pair of longitudinal, flat, transparent side walls, a curved base connecting the bottom edges of the pair of flat side walls parallel to each other, a pair of rounded, corner-less end walls, connecting and sealing the side edges of the pair of flat side walls, an air tube extending along said curved base, a lid situated on the top edges of the pair of flat side walls and an outlet formed in one end of the curved base wall for drainage of the bioreactor, and a blowing means for providing air flow, wherein the blowing means provides the air flow through a medium in said bioreactor, for developing a vortex in the medium containing the algae, thereby preventing sinking of Algae and causing them to be uniformly exposed to light.

### FIELD OF THE INVENTION

The present invention relates generally to Photosynthetic organisms, such as Algae and micro-algae cultivation systems, and more particularly to photobioreactor (PBR) Algae cultivation systems.

### BACKGROUND

Algae are a group of relatively simple, plant like organisms, most of which are capable of performing photosynthesis: They capture light and use its energy to convert CO₂ into sugars and oxygen. In this way, they largely contribute to the global oxygen production (between 50 to 87 percent). There are 80,000 to 100,000 different algae species with widely varying characteristics. Algae size ranges from micrometers of unicellular micro-algae to macro-algae seaweeds of tens of meters. Globally, there is growing interest in algae as production organisms. Algae contain lipids (oil), proteins and carbohydrates (sugars), and especially marine algae have been used as food and fertilizers for centuries. Commercial farming of algae has a long history. Commercial large-scale cultures of the micro-algae species have started in the early 1960s. Nowadays, approximately 200 species of micro-algae are used worldwide. There is a well-established global market for algae-based food and feed products, but microalgae have other functions. More recently, algae are used for the production of ethanol (fermentation) or biodiesel (conversion), and research using GM algae for the production of pharmaceuticals is currently on-going.

Algae in general and single cell micro-algae in particular, represent an important layer of the ecological system, both by their prominent function in the food chain and in absorption of carbon dioxide from the atmosphere and oxygen release.

The Algae chemical composition is characterized in the abundance of poly-unsaturated fatty acids (PUFA), unique proteins and toxins, a variety of polysaccharides and pigments. Single cell Algae grow and multiply at a faster rate than other plants (20-30 times faster).

Currently, systems for commercial cultivation of algae can be divided into three types:

1. Open systems, such as raceways and ponds

2. Closed systems, such as photo-bio-reactors (PBR)

3. Semi-closed systems, such as poly-ethylene sleeves.

### Algae Growth in Open Systems

Until the year 1940, the growth of micro-Algae had been done only by scientists and in labs. During the end of the 1940's, experimentation had started with micro-Algae growth outside labs, as a branch of advanced agriculture, and as food for humans and feeding animals. An additional field of interest focuses on the benefit derived from their ability to exchange gasses and produce Oxygen, and lower the emission of Carbon Dioxide.

Open artificial systems for Algae growth, as opposed to natural water sources, are usually built out of asphalt, concrete, plastic sheets, rubber or foam, so that cleaning can be done efficiently. The system may be shaped as a pool, container or elliptical track that the Algae culture is swirled in by air blowing or by a mixing paddle.

### The Advantages of Open Systems:

Simple, easy and cheap to operate (both in setting up and operation costs)
Easy cleaning and disinfecting after use
Do not require complicated and expensive control systems
Ideal system for manufacturing of cheap products for high-volume markets
Open systems have been in commercial use since the 1950's, therefore there is a lot of experience and knowledge regarding setting up and operation

### The Disadvantages of Open Systems:

Low lighting area to volume ratio—the pools must be shallow to enable enough light to penetrate the culture
Low photosynthetic efficiency (of approximately 1.5%)
Low culture density (0.1-0.2 g per L)
Low production efficiency
Wasteful in land utilization—the volume of the culture may be extended only by two-dimensions, but not by depth
Difficulty in acquiring a stable culture, such that cultivation cannot continue through an extended amount of time
The system is compatible with only a few types of micro-Algae
Waste of resources, such as water evaporation and vaporization of CO₂ into the atmosphere
Exposure to the surroundings and high contamination risk and collapsing of the culture

### Algae Growth in Closed Systems

The disadvantages of the open systems have led to the development of closed Photo Bio Reactors (PBR). In Europe of the 1970's, both closed and open systems were used to grow micro-Algae for the production of Methane. It was then found that cultivation in closed systems is essential in producing high-valued products. The designing of PBR's which achieve high production ability on the one hand, while reducing the production costs, on the other hand, is not trivial. There are many different types of PBR's.

### Advantages of Closed Systems:

Allows optimization and maximal control of the cultivation conditions
High density cultures (2-8 g/L)
High lighting area to volume ratio
High harvesting efficiency due to the low volume and high density
High photosynthetic efficiency (up to 5%)
Allows for a stable and continuous production
Highly economical in land space, CO₂ and water (medium)
Due to the system not being exposed, there is a low contamination rate
The systems are adaptable to many different species of micro-Algae, since they are minimally weather-dependent.
Are able to be cleaned and disinfected by automated systems

### Disadvantages of Closed Systems:

Complicated and expensive to set up and operate
The systems are sensitive. In order to avoid culture collapsing, there is a need for meticulous control over many parameters
Difficult to clean and disinfect
Require higher swirling rates
The conditions are not equal throughout the system (pH gradient, accumulated pressure, accumulated Oxygen)

### Algae Growth in Semi-Closed Systems

For small-scale cultivation of micro-Algae, hybrid systems are used which can be described as semi-closed systems. These are containers for disposable use such as, sleeves, panels, baths or plastic sheets, and are exposed to the external surroundings. These systems are usually used with artificial lighting in a clean room, or exposed to sunlight but protected from the environment, such as in a greenhouse.

### Advantages of Semi-Closed Systems:

Low cost set up, since the containers are disposable and they do not require cleaning
Provide greater protection of the culture than open systems
Do not create positive air pressure or accumulate Oxygen

### Disadvantages of Semi-Closed Systems:

Are not suited for large scale and commercial cultivation
are not suited for continuous growth and production
Wasteful in gasses (CO₂) and work for harvesting
Do not provide appropriate protection from contaminates
   The following table is a comparison between the different cultivation methods:

| | | | | Existing |
|---|---|---|---|---|
| | Tubular | Open | Vertical | Flat Panels |
| | Systems | Ponds | Sleeve | Systems |
| | (Closed | (Open | Systems | (Closed |
| | system) | system) | (Semi-closed) | system) |
| Land | High | low | Medium | Medium-High |
| Utilization | (particularly | | | |
| | in vertical | | | |
| | PBRs) | | | |
| Biomass | High | Low | Medium-High | Medium-High |
| Variety of | Medium | Low | Medium-high | Medium-High |
| Algae species | | | | |
| Contamination | Low * | High | Medium | Medium |
| Potential | | | | |
| Setting-up | High | Low | Medium-Low | Medium |
| Expenses | | | | |
| Operating | High | Low | High | Medium |
| expenses | | | | |
| Maintenance | High | Low | Medium | Medium |
| ease | | | | |
| CO₂ | High | low | Medium | Medium-High |
| | (particularly | | | |
| | in vertical | | | |
| | PBRs) | | | |

* Once the PBR is contaminated it is very difficult to clean it and recover from the contamination

When cultivation methods in the existing Hat Panels Systems are examined, a number of obstacles are presented that need to be overcome in order to maximize the system operation for commercial use. Among the obstacles are:

1. Setting-up and operating expenses
2. System cleaning
3. Up-scaling ability
4. Dismantling and mobility

The growth system of the present invention, it being a closed-flat panels system, overcomes the above obstacles, by possessing the advantages of both closed and open systems.

### SUMMARY OF THE INVENTION

Accordingly, it is a principal object of the present invention to overcome the limitations of prior art Algae growth systems by providing an Algae growth system which combines the advantages of closed systems with the simplicity and low costs of open systems, said system comprising:

a transparent bioreactor device comprising:
a pair of longitudinal, flat, transparent side walls,
a curved base connecting the bottom edges of said pair of flat side walls parallel to each other;
a pair of rounded, corner-less end walls, connecting and sealing the side edges of said pair of flat side walls;
an air tube extending along said curved base;
a lid situated on the top edges of said pair of flat side walls; and
an outlet formed in one end of said curved base wall for drainage of said bioreactor device,

and a blowing means for providing carbon dioxide enriched air,

wherein said blowing means provides air flow through a medium in said bioreactor device, for developing a vortex in the medium containing the algae, thereby preventing sinking of algae and causing them to be uniformly exposed to light.

According to a preferred embodiment of the present invention, the photo bioreactor (PBR) device comprises a transparent aquarium with longitudinal flat side walls, a curved base, rounded end walls without corners and a lid that can be opened. The PBR device is filled with a cultivation medium and is vortexed by air flowing through a tube having holes, in the bottom of the aquarium. The aquarium is constructed in such a way that the internal side is smooth and there are no corners, edges, or rough surfaces that elevate friction and are prone to adherence of biofilm or contaminating aggregates, which are usually very difficult to clean and sterilize. By having a rounded and smooth construction, contamination is avoided.

The PBR device base is composed of a rigid material that is curved according to the varying PBR device width. The curved base results in a uniform water flow in the entire width of the device, which prevents algae from settling on the bottom and on the walls of the PBR device. Furthermore, it allows for a large width of the PBR device to remain without algae settling or sinking therein, and there is no need for a lot of air for appropriate movement of the algae. The rounded base further allows easy cleaning and draining.

A drain placed on the other side of the base allows for fast harvesting which provides smaller chances of the Algae to decompose or rot.

For the purpose of continuous growth, when only part of the Algae are harvested and the rest remain in the PBR device, and growth medium is added for continued growth, the drain is especially useful. During continuous growth, after a while the Algae begin to grow on the walls, then a wiper can be wiped against the walls and remove the Algae from them. This can be repeated frequently when the Algae begin growing on the walls again.

The device is modular which provides a significant advantage in commercial cultivation.

In accordance with a further embodiment of the present invention, there is provided a PBR device with an air tube placed externally to the PBR device. The external air tube has a laterally extending slit, resulting in a U-shaped tube. Slit air tube is attached on its opened end to the curved base. The curved base has holes created thereon, to allow the CO2 blown from within the air tube to enter the PBR device. This construction minimizes accumulation of dirt, and is easy to clean. In addition, there is no limit to what the length of the tube can be, and it eliminates problems existing with the internal air tube, such as the tube becoming lifted and having to glue the tube to the base, which causes contamination.

In accordance with yet a further embodiment of the present invention, there is provided a method for external temperature control by using a wet cloth blanket, or an equivalent, when the blanket is soaked in water at a desirable temperature, thus heat transfer is created between the wet blanket and the PBR device growth medium.

In accordance with an additional embodiment of the present invention, there is provided another method for external temperature control by using a double-jacket, which is double-walled with water or cooling liquid directed to flow therethrough at a desirable temperature. The double-jacket is wrapped around the PBR device, either on both sides or only on one side. The double jacket creates heat transfer between the cooling liquid in it and the liquid inside the PBR device. The double jacket may be transparent or opaque.

In accordance with further embodiments of the present invention, there are provided yet other methods for external temperature control by soaking the system in a pond of water, outer water sprinklers, an inner heat exchange coil flowing with temperature-controlled water, placed within the PBR device, and a plastic tent covering the entire PBR system having air conditioning to control the temperature. Inner heat exchange coil may be removed from the PBR device to be cleaned and sterilized.

In accordance with yet another further embodiment of the present invention, there is provided a sun tracking system on the PBR device, which causes the PBR devices to tilt towards the sun, thereby increasing the exposure to the sunlight.

By connecting several devices to one another into one growth unit, more benefits are provided, such as lowering the operational expenses.

The system is created by connecting several devices to one another at either end of the device via a hose positioned perpendicular to the bottom of the device. Alternatively, the devices may be connected to each other by placing a water-filled connector aquarium perpendicular to the devices. Each device is connected to the connector aquarium via openings in the connector aquarium, which creates a manifold on one side or both.

The device can work anywhere in the world, and can be used with natural or artificial light, such as, but not only, Fluorescence and LED.

Additional features and advantages will become apparent from the following drawings and description.

### BRIEF DESCRIPTION OF THE DRAWINGS

For a better understanding of the invention with regard to the embodiments thereof, reference is made to the accompanying drawings, in which like numerals designate corresponding elements or sections throughout, and in which:

FIG. 1 shows a Flat Panel Photo-Bioreactor (PBR) device, according to an embodiment of the present invention;

FIG. 2 shows a side view of the PBR device of FIG. 1;

FIG. 3 shows a prospective view of the frame of the PBR device of FIG. 1;

FIG. 4 shows the PBR device of FIG. 1 without the frame and lid;

FIG. 5 shows a bottom view of the PBR device of FIG. 1;

FIG. 6 shows the PBR device of FIG. 4 with a lid;

FIG. 7 shows an alternative embodiment of the PBR device;

FIG. 8 shows another alternative embodiment of the PBR;

FIG. 9 shows a perspective view of a cooling pond for multiple PBR devices; and

FIG. 10 shows a perspective view of a system of multiple rows and columns of PBR devices.

### DETAILED DESCRIPTION OF THE PREFERRED EMBODIMENTS

It is a principal object of the present invention to provide a modular Flat Panel Photo-Bioreactor system for cultivating many different types of algae, which provides a significant advantage in commercial cultivation.

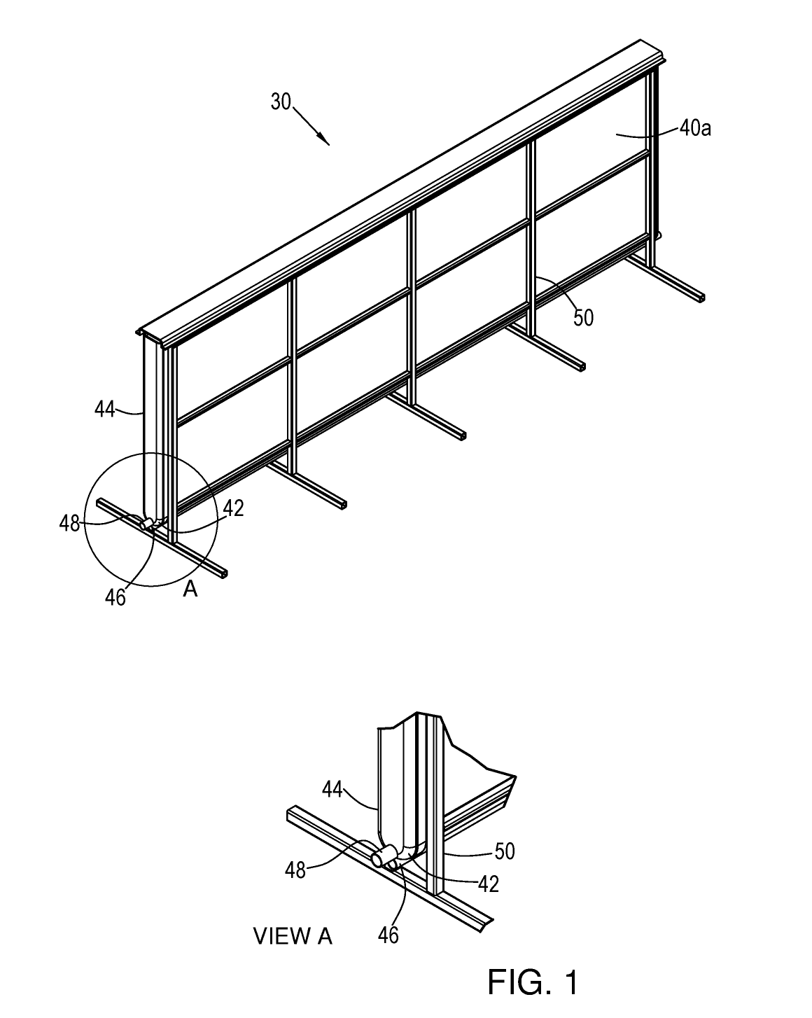

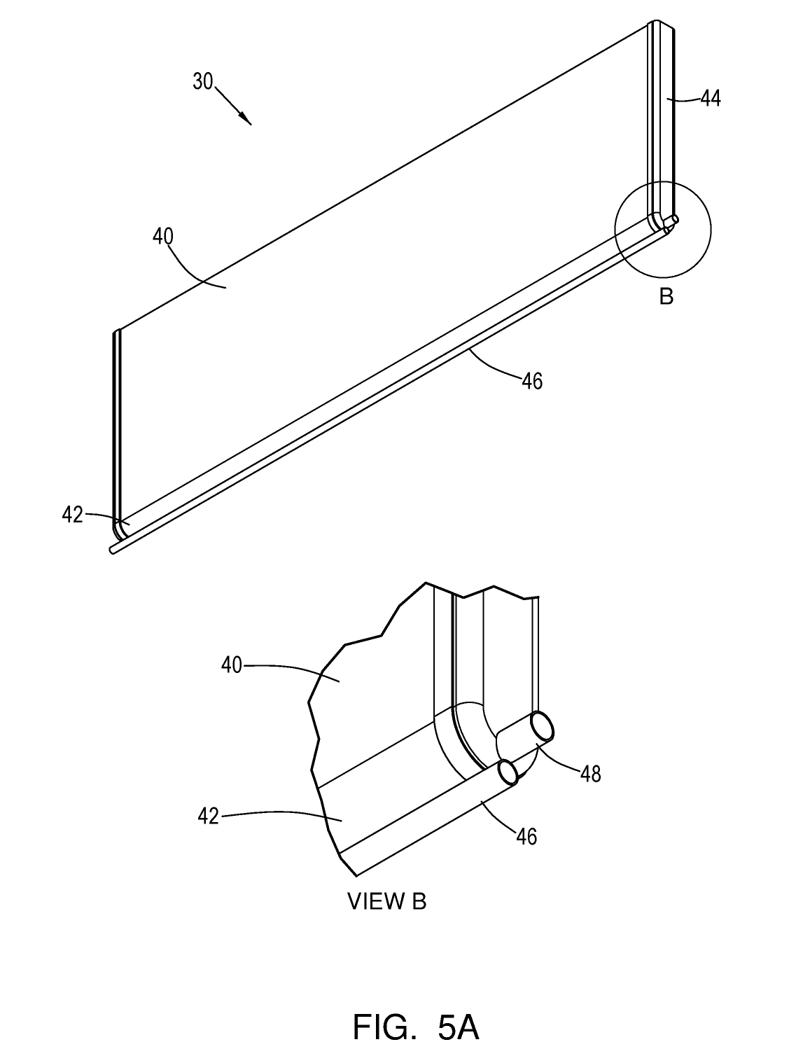

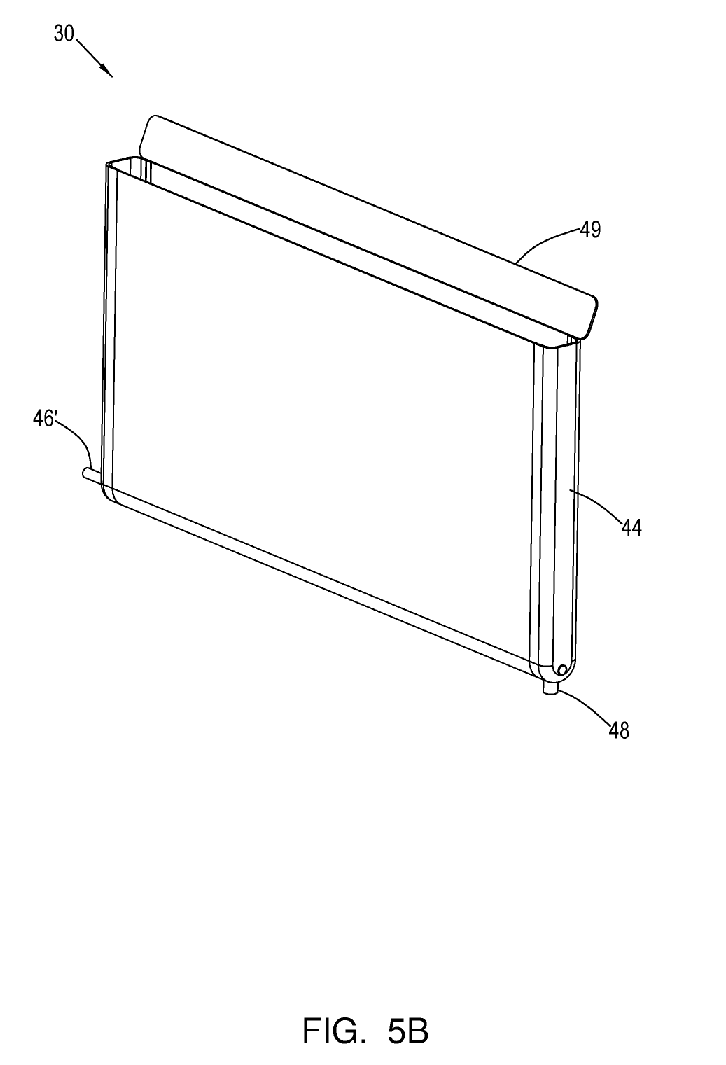

Referring now to FIG. 1, there is shown a Flat Panel Photo-Bioreactor (PBR) device 30 comprising a pair of longitudinal, flat, transparent walls 40a, 40b (not shown here), positioned parallel to one another, and a curved base 42 connected to the bottom edges of walls 40a-b. A pair of rounded corner-less side walls 44a-b are provided for connecting the side edges of walls 40a-b. An air tube 46 is positioned at the base 42, perpendicular to walls 40a-b, either internal to the PBR device 30 (shown in FIG. 5B) or external to the PBR device 30. A lid 49 is placed on the top edges of walls 40a-b, and may be opened or closed. Lid 49 is installed on an axis so that it remains attached to the device 30 also when it is opened, and allows operation of the device also when opened. For the purpose of draining device 30, there is provided an outlet 48 positioned parallel to air tube 46.

Longitudinal flat side walls 40a-b are made of rigid transparent plastic with a thickness in a range of 0.5-12 mm, a height in a range of 30-300 cm or more, width in a range of 3-40 cm and a length in a range of 0.5-1000 m. The walls may be constructed of any transparent material, such as glass or plastic.

PBR device 30 is closely surrounded by frame 50 which maintains PBR device 30 in its position, and further prevents walls 40 from becoming distorted due to the positive hydro-pressure created in PBR device 30. Walls 40 are plastic which has elasticity that causes walls 40 to be prone for distortion.

On end wall 44 there is formed a rounded closure laterally extending to base 42 without angles. Air tube 46 extends through one end wall 44 situated at a height of 0.5-5 cm from the bottom, running all along the length of PBR device 30. Tube 46, having a diameter of 16-60 mm, has holes in the size of 0.5-5 mm, 1-20 cm apart from each other, directed upwards in order to blow Carbon Dioxide-rich air into the medium for the growth of the Algae, and also for causing vortexing motion of the water to avoid algae sinking, flocculation and aggregation, and for providing uniform exposure of light to all the algae. Walls 44 also allow for harvesting algae within the device 30 and high quality cleaning and disinfecting the device 30.

For economizing purposes air tube 46 may blow air that is not enriched with CO₂ into PBR device 30. In this case, CO₂ is delivered into PBR device 30 via a separate tube inserted through a separate opening in PBR device 30, or by placing in PBR device 30 a ceramic stone, or other Carbon sources such as, but not only, bicarbonate, glycerol or sugar.

A lid 49 is placed on the top of PBR device 30 to provide a sterile environment by preventing contaminants from entering PBR device 30 by creating a positive hydro-pressure in the PBR device 30. Lid 49 is composed of rigid cleanable material which is transparent to allow light to pass through it. Lid 49 also minimizes the CO₂ intake, allows for working with an open or closed device, contributes to temperature homeostasis according to weather, allows for an option for different types of cooling and allows easy cleaning and a possibility for automation. There is less medium evaporation, no medium spraying and rain cannot enter the device 30.

The PBR device 30 walls are corner-less and made of rigid materials so that the walls are smooth without creases, folds, or “dead” areas, a feature which provides a large surface-area that provides a large light exposure. Due to these features, there is substantially no Algae growth on the walls which are easily cleaned and sterilized.

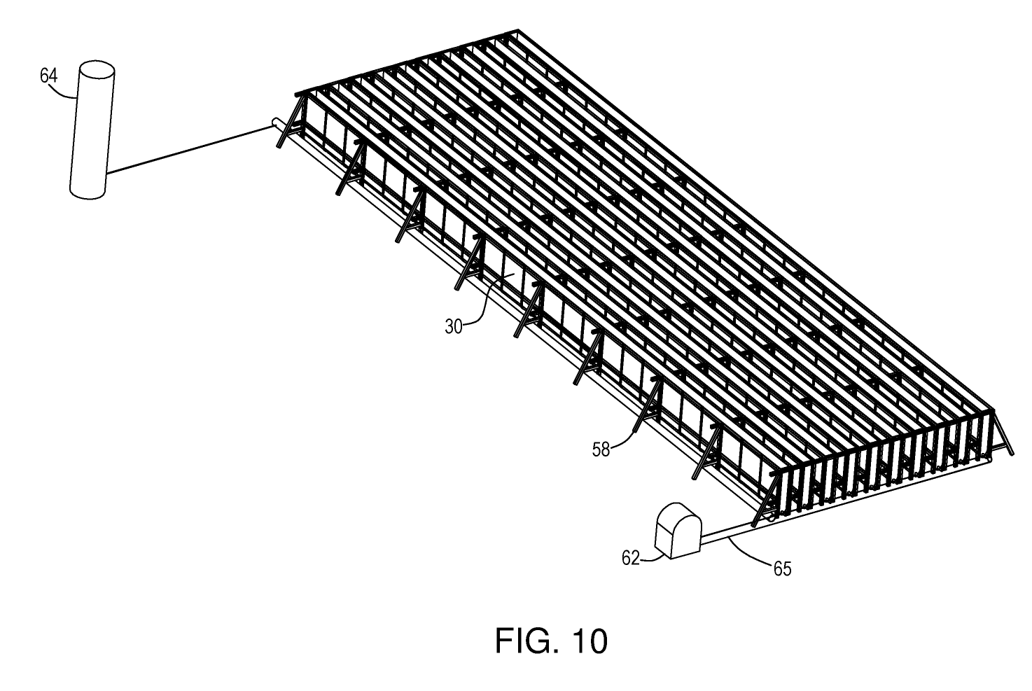

Air tube 46 is connected to one end of a blower which is connected on its other end to a CO₂ tank (shown in FIG. 10), and tube 46, which is internal to PBR device 30, extends along base 42 of PBR device 30, and blows CO₂-rich air into the medium. Tube 46 contains multiple holes which allow for homogenous air release which creates the water movement, thereby resulting in a large exposure of light and a large biomass growth. The air blowing into the base creates an area in the PBR device 30 base onto which the Algae cannot settle on.

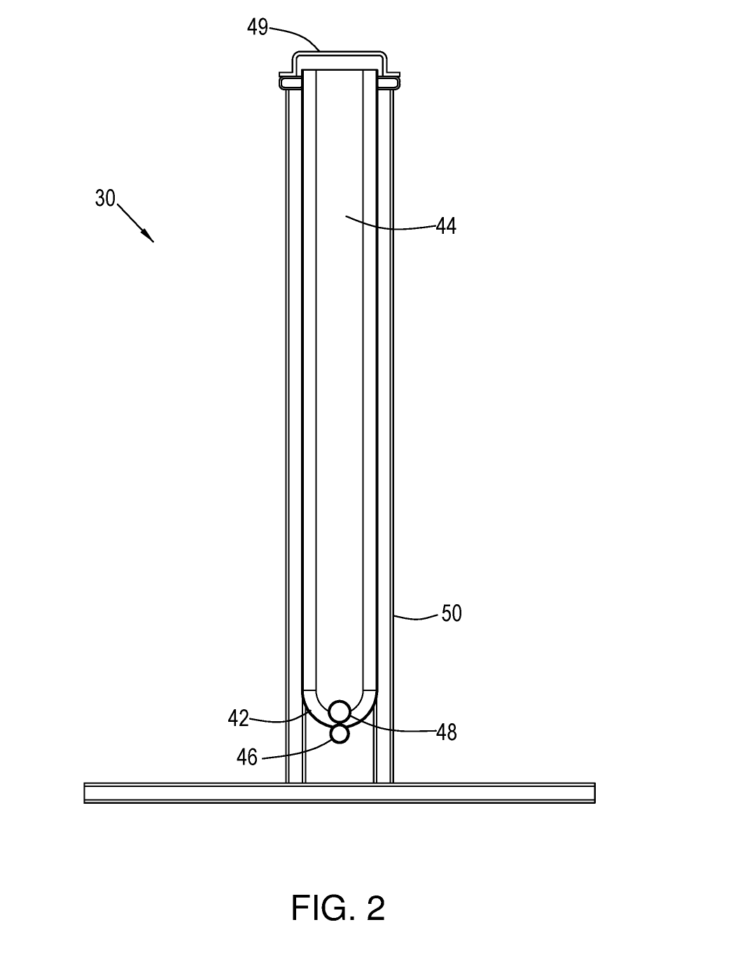

Referring now to FIG. 2, there is shown a side view of the device of FIG. 1 showing air tube 46 external to PBR device 30, and parallel to outlet 48.

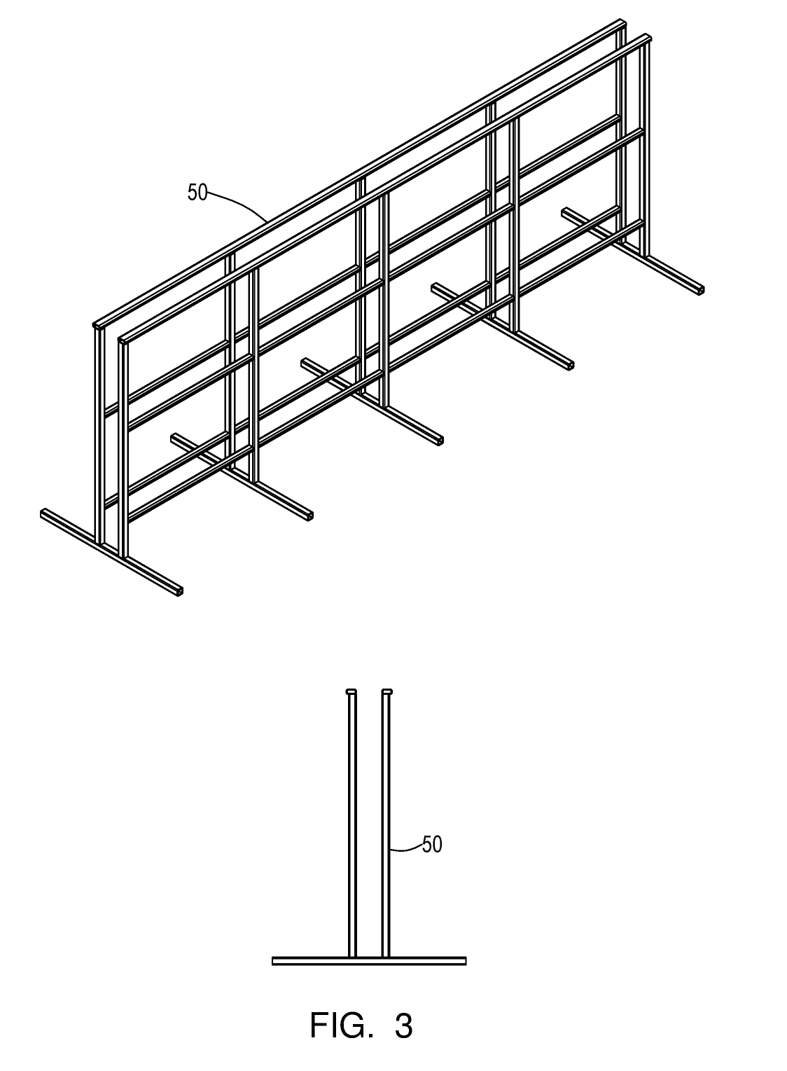

Referring now to FIG. 3, there is shown perspective and side views of frame 50 without PBR device 30.

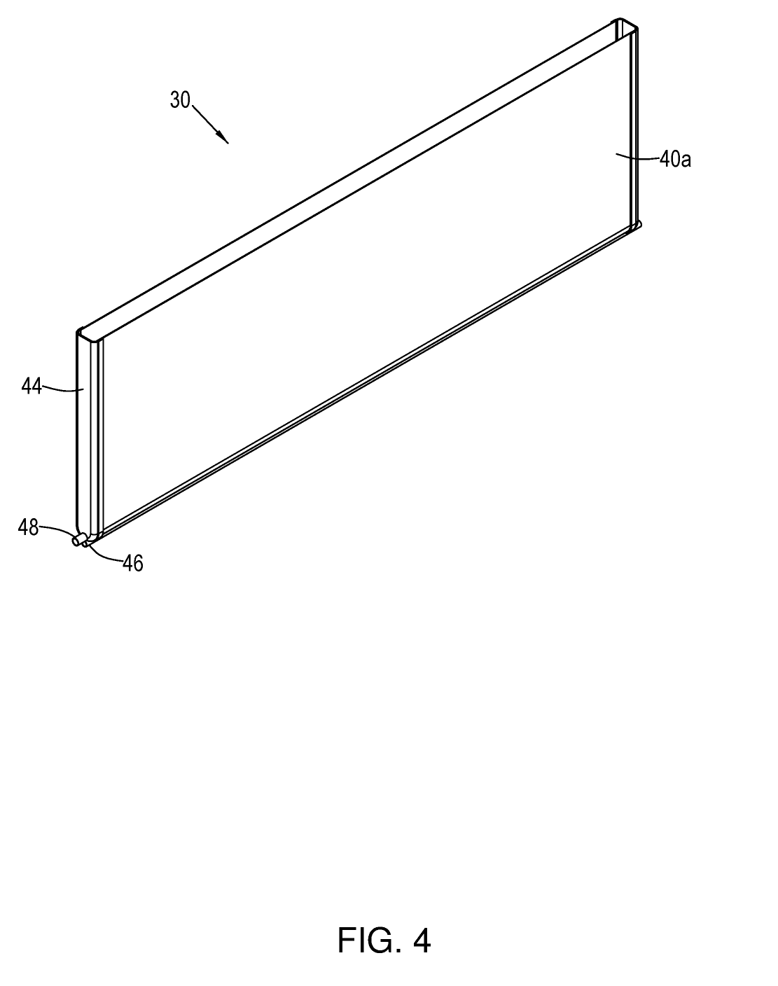

Referring now to FIG. 4, there is shown a PBR device 30 without frame 50 or lid 49. Although the previous embodiments shown in FIGS. 1-3 describe the construction of PBR device 30 as an assembly of elements, it is possible to manufacture a PBR device 30 as a single integral unit. This can be achieved, in the case of plastic, by extrusion molding, and in the case of glass using suitable glass-shaping techniques.

Referring now to FIG. 5A, there is shown a bottom view of PBR device 30, illustrating that air tube 46 is external to PBR device 30 and is attached to curved base 42. View B is an enlargement of the ends of air tube 46 and outlet 48, which are parallel to each other.

Referring now to FIG. 5B, there is shown PBR device 30 without frame 50, having air tube 46′ internal to PBR device 30. Air tube 46′ extends through one end wall 44 situated at a height of 0.5-5 cm from the bottom, running all along the length of PBR device 30. Tube 46′, having a diameter of 16-60 mm, has holes in the size of 0.5-5 mm, 1-20 cm apart from each other, directed upwards in order to blow Carbon Dioxide-rich air into the medium for the growth of the Algae, and also for causing vortexing motion of the water to avoid algae sinking, flocculation and aggregation, and for providing uniform exposure of light to all the algae. Walls 44 also allow for harvesting algae within the device 30 and high quality cleaning and disinfecting the device 30. The air blowing into base 42 creates an area in the PBR device 30 base onto which the Algae cannot settle.

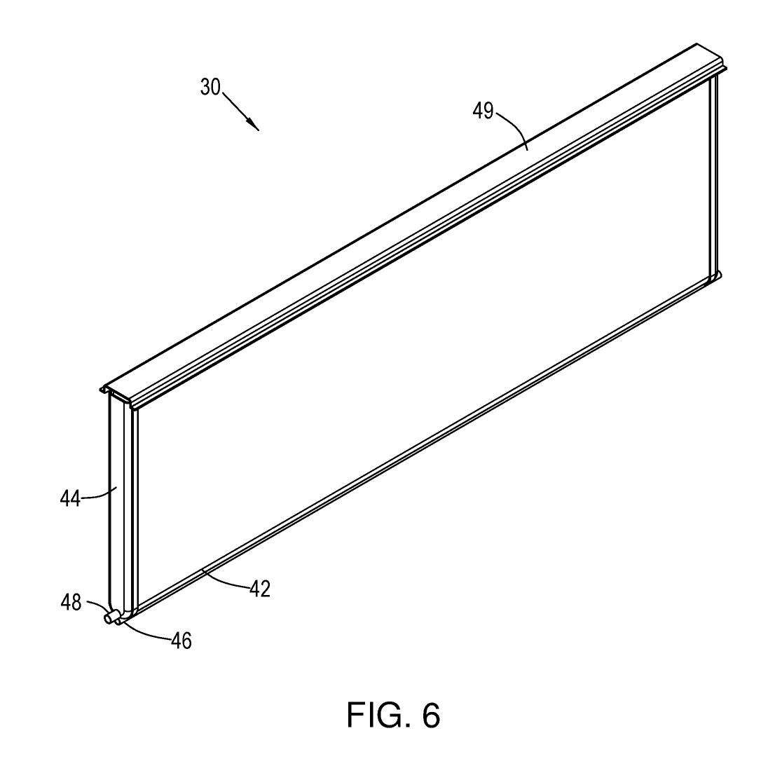

Referring now to FIG. 6, there is shown PBR device 30 having lid 49 sealed thereon, thereby protecting the Algae and cultivation medium within PBR device 30 from possible contaminates, and additionally creating positive air hydro-pressure within PBR device 30 by preventing external air from entering PBR device 30.

Lid 49 may be constructed of any material such as rigid plastic, soft plastic (such as a bag), or any other suitable material, and can be either transparent or opaque. Lid 49 may be attached to PBR device 30 by hinges, or by simply placing lid 49 on PBR device 30, or by any other suitable configuration.

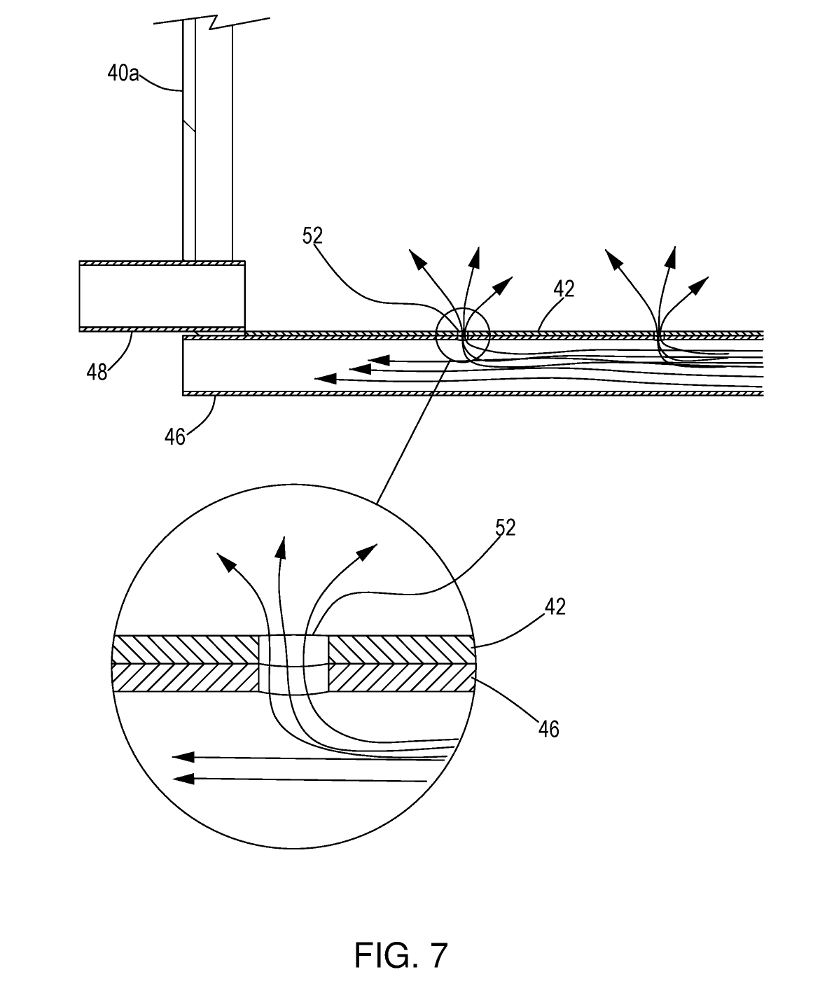

Referring now to FIG. 7, there is shown air tube 46 externally attached to curved base 42, allowing air (marked by arrows) to enter PBR device 30 by creating holes 52 in base 42 that are respective to holes created in air tube 46. Air tube 46 may be glued to base 42. When tube 46 is internally mounted in PBR device 30, it is glued to base 42, and the glue often causes contamination. In addition, when tube 46 is internally mounted, it tends to become lifted due to buoyancy of the air-filled tube 46. Therefore, having air tube 46 externally mounted helps prevent such contamination and avoids the buoyancy problem.

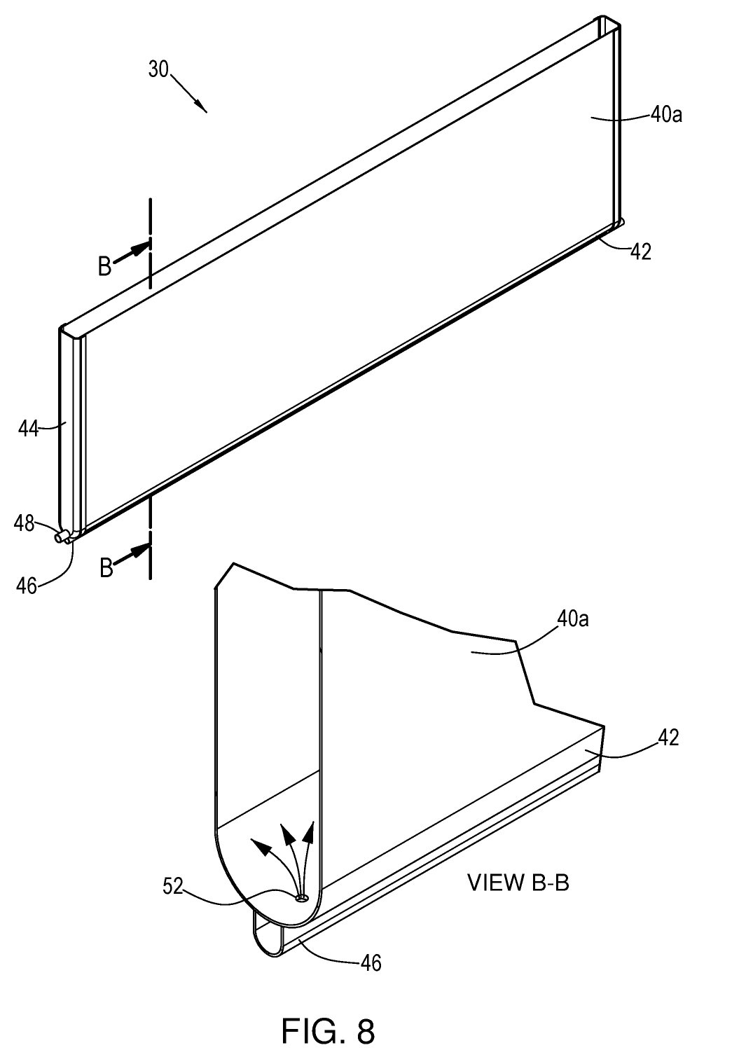

Referring now to FIG. 8, there is shown air tube 46 external to PBR device 30, as shown in FIG. 1, air tube 46 has a laterally extending slit, resulting in a U-shaped tube. Slit air tube 46 is attached on its opened end to curved base 42. Curved base 42 has holes 52 created thereon, to allow the CO₂ blown from within air tube 46 to enter PBR device 30. This construction minimizes accumulation of dirt, and is easy to clean.

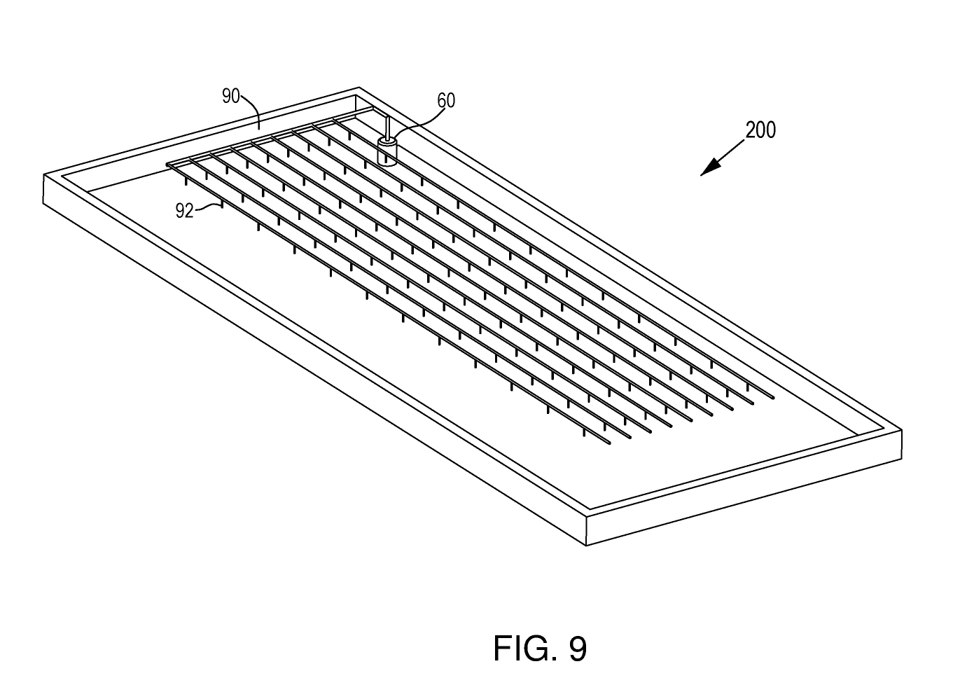

Referring now to FIG. 9, there is shown cooling system 200 which is a plurality of rows and columns of PBR device 30, situated in a cool water pond, so that there is no need for injecting cooling water into the aquarium, and the cooling is done only externally.

There is provided a water pump 60, which is a source of water for spray system 90 situated above the plurality of PBR devices 30 (not shown). Spray system 90 has multiple spray nozzles 92 positioned between each device 30, for the purpose of cooling the water in PBR devices 30. Spray system 90 may also function as the source of water for filling a double jacket, and moistening a blanket, both for the purpose of cooling the water in PBR device 30.

The same system 200 can be used for heating as well for heat mass transfer, by filling the system with hot/warm water instead of cold water.

Referring now to FIG. 10, there is shown multiple PBR devices 30 which are positioned in a plurality of rows and columns, and may or may not be connected to each other. Air blower 62 which is connected to manifold 66 via connecting pipe 65, and blows air into manifold 66 which blows air into each PBR device 30. CO₂ tank 64 is connected to connecting pipe 65, before manifold 66, and enriches the air that enters devices 30 with CO₂. The air blown into the water is for allowing constant circulation of the algae in the water, which provides uniform light exposure to all the algae cells, and prevents sinking and wall growth, and this prevents rotting of the algae and contamination.

Although particular embodiments of the invention have been described and illustrated herein, it is recognized that modifications and variations may readily occur to those skilled in the art and consequently, it is intended that the claims be interpreted to cover such modifications and equivalents.

1. A system for algae growth, said system comprising:
a transparent bioreactor device comprising:
a pair of longitudinal, flat, transparent side walls,
a curved base connecting the bottom edges of said pair of flat side walls parallel to each other;
a pair of rounded, corner-less end walls, connecting and sealing the side edges of said pair of flat side walls;
an air tube extending along said curved base;
a lid situated on the top edges of said pair of flat side walls; and
an outlet formed in one end of said curved base wall for drainage of said bioreactor device,
and a blowing means for providing air flow,
wherein said air tube is external to said bioreactor, and wherein said air tube comprises a laterally extending slit resulting in a U-shaped tube, wherein said U-shaped tube is attached at its open end to said curved base, and said curved base has spaced-apart holes formed thereon, for allowing air blowing within said air tube to enter said bioreactor via said holes on said curved base, and
wherein said blowing means provides said air flow through a medium in said bioreactor device, for developing a vortex in the medium containing the algae, thereby preventing sinking of algae and causing them to be uniformly exposed to light.

2. The system of claim 1, wherein said pair of longitudinal flat transparent walls are made of at least one of transparent plastic and glass, with a thickness in a range of 0.5-12 mm, a height in a range of 30-300 cm or more, a width in a range of 3-40 cm and a length in a range of 0.5-1000 m.

3. The system of claim 1, wherein said air tube is internal to said bioreactor device, attached to said curved base, and wherein said air tube comprises a plurality of holes for the homogenous release of said carbon dioxide enriched air, for the purpose of creating an area in said curved base in which the algae cannot settle.

4. The system of claim 1, wherein said air tube has a diameter of approximately 16-60 mm and said holes have a diameter of approximately 0.5-5 mm.

5. The system of claim 1, wherein said air tube is external to said bioreactor and attached to said curved base, wherein said air tube comprises a plurality of holes, wherein said holes are aligned to match respective holes formed in said curved base, thereby allowing air blowing within said air tube to enter into said bioreactor via said holes on said curved base.

6. (canceled)

7. The system of claim 1 wherein said transparent bioreactor comprises a frame closely surrounding said bioreactor, wherein said frame maintains said bioreactor in its upright position and prevents said side walls from becoming distorted due to positive-hydro pressure created within said bioreactor.

8. The system of claim 1 wherein said lid comprises a rigid cleanable material that is transparent for allowing light to pass through it, wherein said lid allows for said system to operate when it is either closed or open, and wherein said lid prevents contamination of said bioreactor and allows for positive hydro-pressure to be created inside said bioreactor.

9. The system of claim 8, wherein said lid can be made of soft material and any other suitable material, and may be opaque, and further may be attached to said bioreactor by hinges.

10. The system of claim 8, wherein said lid may be attached in sealing fashion to said bioreactor by placing it over said bioreactor.

11. The system of claim 1, wherein said blowing means comprises an air blower that is connected to a manifold which is connected to said air tube, and wherein said air blower is connected to a CO2 tank.

12. The system of claim 1, further comprising a cooling system, said system comprising:
a plurality of bioreactors arranged in an array of rows and columns;
a cool water pond; and
a spray system, comprising spray nozzles;
wherein said plurality of bioreactors are situated in said cool water pond for the purpose of cooling said bioreactors to the temperature of said cool water pond, and wherein said spray nozzles provide the cool water said cool water pond.

13. The system of claim 1, wherein said bioreactor is wrapped by a double jacket for the purpose of external temperature control, wherein said double jacket comprises a double wall filled with flowing temperature-controlled liquid at a desirable temperature.

14. The system of claim 1, wherein said bioreactor is wrapped by a wet blanket comprising an absorbing material soaked in liquid at a desirable temperature, for the purpose of creating heat transfer between said wet blanket and said bioreactor.

15. The system of claim 1, wherein said bioreactor comprises an inner heat exchange coil between said side walls, wherein said coil flows with temperature controlled liquid, wherein said coil can be removed for cleaning and sterilizing and can be one of transparent and opaque.

16. The system of claim 1, further comprising a plurality of said bioreactors, wherein said bioreactors are connected to one another at either end via a hose positioned perpendicular to said bottom of said bioreactor.

17. The system of claim 1, wherein said system comprises a plurality of said bioreactors, wherein said bioreactors are connected to one another at either end by placing a water-filled connector aquarium perpendicular to said bioreactors, wherein each said bioreactor is connected to said connector aquarium via openings in said connector aquarium, which creates a manifold on one side or both.

18. The system of claim 1, further comprising a sun tracking system which causes said bioreactors to tilt towards the sun, thereby increasing the exposure to the sunlight.

19. The system of claim 1, wherein said bioreactor is manufactured of plastic as a single plastic integral unit.

20. The system of claim 1, wherein said bioreactor is manufactured of glass as a single glass integral unit.

21. The system of claim 1, wherein said blowing means provides carbon dioxide enriched air.

22. The system of claim 1, wherein said system further comprises a carbon source for growth of the algae, such as, but not limited to, a carbon stobne, bicarbonate, glycerol and sugar.
